(19) United States
(12) Patent Application Publication
(10) Pub. No.: US 2008/0218695 A1
(43) Pub. Date: Sep. 11, 2008

(54) Optical device, use of an optical device according to the invention as well as method for blocking light reflections in the observation beam path of an optical device

(76) Inventor: Andreas Obrebski, Oberkochen (DE)

Correspondence Address: KRIEGSMAN & KRIEGSMAN, 30 TURNPIKE ROAD, SUITE 9, SOUTHBOROUGH, MA, 01772, US

(21) Appl. No.: 11/897,009

(22) Filed: Aug. 28, 2007

(30) Foreign Application Priority Data
Aug. 21, 2006 (DE) 10 2006 039 137.3

(51) Int. Cl. A61B 3/12

(52) U.S. Cl. 351/213

(57) ABSTRACT

The invention relates to an optical device for the examination of an eye, with at least one objective disposed facing the eye, at least one lighting source for illuminating the eye, an optical element and/or an optical arrangement disposed between the objective and the eye being examined, which optical element or arrangement produces an intermediate image of the plane examined within the eye, which is observed with the objective, whereby in the observation beam path of the optical device, at least one locally resolved attenuating element is disposed, which can be switched in a locally resolved manner into at least two transparency states. In addition, the invention relates to a method for blocking light reflections in the observation beam path of an optical device as well as the use of such an optical device for examining an eye.

The present invention relates to an optical device, in particular an opthalmoscope, for observing an eye, in particular the fundus of an eye. In addition, the invention relates to a method for blocking light reflections in the observation beam path of an optical device as well as the use of an optical device according to the invention.

Opthalmoscopy or funduscopy is used for the evaluation of pathologic changes in the portion of an eye that can be examined. In an eye examination, the observer looks through the pupil of the eye into the inside of the eye by means of an optical device having an optical element, in particular a lens. For this purpose, the eye must be illuminated with a light source. Two different kinds of opthalmoscopy can be distinguished.

In so-called direct opthalmoscopy, an opthalmoscope is brought up very close between the eye being examined and the eye of the observer. The distance between the eye of the observer and the eye being examined is approximately 100 mm, so that the examination is often perceived as unpleasant, particularly for the patient. In direct opthalmoscopy, the central parts of the eye, such as vessel origins or the optic disk can be observed simply and clearly enlarged.

In so-called indirect opthalmoscopy, the background or fundus of the eye is observed from a distance of approximately 150-200 mm by employing a light source and an optical element, in particular a lens, which is kept approximately 10-15 mm in front of the eye to be examined. In this case, the retina, the periphery of the retina, the optic nerve, the vessels as well as the macula lutea are readily examined. The magnification is smaller than in the case of direct opthalmoscopy. The overview of the eye is essentially better when compared with direct opthalmoscopy, and a stereoscopic (3D) observation of the eye is possible.

In the case of indirect opthalmoscopy, the examination of the fundus of the eye may also be conducted with a slit lamp. In the case of a slit-lamp examination, the retinal image can be enlarged or can be evaluated under projection of a slit lamp. A slit lamp is fastened to a microscope for the slit lamp examination. The slit lamp emits a narrow, slit-form light bundle. The constricted light bundle of the slit lamp makes possible an optical sectioning through the transparent segments of the eye tissue. Then the fine structure, position and thickness of the tissue can be well recognized. The anterior eye segments such as the cornea, the conjunctiva, the sclera, the iris, the anterior chamber of the eye and the lens can be precisely evaluated with the slit lamp. If certain additional apparatus is connected, such as, e.g., a strong lens, in front of the microscope, then the vitreous body, retina and optic disk can also be examined.

The advantage of indirect opthalmoscopy when compared with direct opthalmoscopy is the greater overview. Of course, the indirect technique requires somewhat more training or experience on the part of the ophthalmologist.

Difficulties also occur, however, in examinations of the ocular fundus by means of the above-described method. For example, light reflections are very disruptive in eye surgeries. Light reflections occur at the optical element, in particular a lens, which is kept in front of the eye to be examined, or at the cornea of the examined eye due to the lighting device of the operating microscope or the lighting of the surgical theater. These light reflections are dazzling to the observer of the eye being examined and in this way adversely affect the result of the examination.

Different devices are known from the prior art for reducing or avoiding light reflections. For example, so-called light traps are known for eye examination equipment from DE 3339172 A1; these prevent the retina of the eye from being loaded with lighting beams. It is disclosed therein that a light-absorbing layer is disposed in a central region of the lighting beam path of an operating microscope in a plane conjugated to the object plane. This light-absorbing layer is designed as the central part of an annular diaphragm opening. The light-absorbing layer (light trap) described in DE 3339172 A1 has the disadvantage that the site of the eye to be examined and covered by the light-absorbing layer is not sufficiently illuminated in order to conduct detailed examinations therein. The eye examination device is also not designed in a flexible manner, so that an appropriate adaptation for different eyes being examined is not possible. An adaptation of the light traps to different corneas or to differently reflected light beams during the examination is not possible.

An ocular microscope is known from DE 19812050 A1, in which the illumination of the eye will be simplified by the use of electronically controllable (relative to their light transmission, light reflection or light emission) chip components that are illuminated by incident light or are transilluminated or are luminescent. In fact, a specific beam of a beam bundle can be blocked by these chip components, but these ocular microscopes also cannot be flexibly adjusted, if light is reflected from different light sources, both from the lens as well as from the cornea of the eye being examined.

EP 1486159 A1 describes an opthalmoscope for the examination of the fundus of an eye with a lens that is disposed between the objective of the opthalmoscope and the eye being examined. On the one hand, the observation beams of the observer, and, on the other hand, the illumination beams of a light source are guided through the lens. The lens has two surfaces on which the beams of the illumination are reflected. In order to prohibit these reflections that are disruptive to the observer, the opthalmoscope of EP 1486159 A1 provides that, first of all, the lens is disposed in a tiltable manner in order to deflect away the reflection or reflections from the objective. This has the disadvantage that the opthalmoscope must be configured in a very complex manner, since a mechanical device must be provided, by means of which the lens can be tilted. Light reflected from the cornea of the eye being observed in turn reaches the observation beam path of the observer and thus disrupts the visual perception of the observer. The lenses used in such an opthalmoscope are designed to be just planar, due to the mechanical requirement of the pivoting device for the lens, and have a maximum of 40 diopters, which is not high enough for many applications.

An optical device known from EP 1486159 A1 for the examination of an eye or the fundus of an eye has at least one eyepiece for observing the eye, at least one other tube containing optical lenses, an objective disposed so that it faces the eye, at least one lighting source for the illumination of the eye, a lens disposed between the objective and the eye to be examined, as well as an interrupt element rigidly disposed in the optical device.

Proceeding from the optical device known from EP 1486159 A1, the object of the present invention is to create an optical device, particularly an opthalmoscope, as well as a method for examining an eye, which does not have the disadvantages of the prior art. In particular, the optical device will prevent light reflections on the optical element or the optical arrangement, which is disposed between an objective of the optical device and the eye being examined, and on the cornea of the eye being examined, from falling in a disruptive manner on the eye of the observer. In addition, the optical device will be adjusted in a flexible manner so that the light reflections of differently curved corneas can be examined.

The object is accomplished according to the invention by an optical device according to patent claim 1, by an optical device according to patent claim 16, by a method according to patent claim 17, as well as the use of such an optical device according to the invention according to patent claim 19. Additional configurations of the invention result from the dependent claims. Other advantages, features and details of the invention result from the subclaims, the description, as well as the drawings. Advantages, features and details, which are described in connection with the optical device according to the invention, apply also, of course, in connection with the method according to the invention as well as the use of the optical device, and vice versa.

An optical device for the examination of an eye, with at least one objective disposed facing the eye, at least one lighting source for illuminating the eye, an optical element and/or an optical arrangement disposed between the objective and the eye being examined, which optical element or arrangement produces an intermediate image of the plane examined within the eye, which is observed with the objective, whereby in the observation beam path of the optical device, at least one locally resolved attenuating element is disposed, which can be switched in a locally resolved manner to at least two transparency states, represents an optical device, in particular an opthalmoscope, for observing the fundus of an eye, which prevents the light reflections on the lens and on the cornea of the eye being examined from falling in a disruptive manner on the eye of the observer. The optical device is adjusted in a flexible manner so that the reflections of differently curved corneas can be considered. A lens system or a magnification system can be provided as an optical element or an optical arrangement. In comparison to the interrupt element known from the prior art in the illumination beam path of an optical device, the attenuating element according to the invention is provided in the observation beam path of the optical device and can be switched to different transparency states in a locally resolved manner as a function of the light intensity of the individual light beams in the observation beam path. That is, the transparency of the attenuating element can be attenuated.

Light reflections can be blocked in a targeted manner by the locally resolved switching possibilities of the attenuating element, into at least two transparency states, in the observation beam path or in the observation beam bundle of the optical device. That is, parts of the reflected lighting beams can be stopped down in the observation beam path of the optical device. The illumination of the eye can remain uniform in such an optical device, while the light reflections on the optical element, particularly on a lens, which is disposed between the objective of the optical device and the eye being examined, and the light reflections on the cornea of the eye being examined in the observation beam path can be blocked, so that these do not fall on the eye of the observer in a disruptive manner. Advantageously, in addition to light beams reflected from the lighting device, such an optical device can also block light beams or reflected light beams from the lighting of the operating room.

Locally resolved means that individual regions of the attenuating element over the cross section of the observation channel can be switched differently. That is, the attenuating element is subdivided into many regions, which can be switched into different transparencies. The cross section of the attenuating element, for example, is equivalent to a matrix with many small squares, which can be controlled individually.

Thus, for example, the attenuating element or individual regions of the attenuating element can be switched between a transparent state and a diffuse state. In the diffuse state, a region of the attenuating element blocks or occludes parts of the beam path, in which the attenuating element is disposed. Thus, the attenuating element takes over the function of partial shutter diaphragms or so-called light traps. Of course, the attenuating element blocks light reflections on the optical element, in particular on a lens, or the cornea of the eye in the observation beam path, and not in the illumination beam paths, as is known in the prior art.

In the transparent state, the attenuating element does not block or the respective locally resolved regions of the attenuating element do not block the observation beam path(s) or parts of the observation beam path(s) of the optical device, so that reflected light beams can pass unhindered through the observation beam path(s) of the optical device into the eye of the observer.

It is advantageous if the attenuating element represents a so-called block matrix. That is, the cross-sectional surface of the attenuating element is divided into a plurality of blocks, whereby each individual block can be switched between at least two transparency states, in particular from a transparent state into a diffuse state. The attenuating element may also be designed in such a way that it has one or more pivotable “pointers”, whereby the end piece or the end pieces of the pointer(s) has/have the size of one or more blocks. Depending on what is needed in each case, the pointer(s) can cover targeted blocks of the attenuating element and thus block parts of an observation beam path.

In the optical device according to the invention, the attenuating element sits on the side of the optical element or the optical arrangement, particularly a lens, which is facing away from the eye. In this way, light reflections which proceed from the two surfaces of a lens, for example, can be simply suppressed. Advantageously, the attenuating element sits directly above the lens. Disruptive light beams, which are reflected on both surfaces of the lens, can thus be directly blocked. In this region directly above the lens, i.e., on the side of the lens facing away from the eye that is being observed, the scatter of the reflected light beams is minimal, so that only a few regions/blocks of the attenuating element must be placed in a diffuse state. The lens itself can be fastened directly onto the eye being examined.

In a preferred embodiment of the optical device, the at least one attenuating element is fastened so that it can be moved along a holder of the optical device. In this way, the attenuating element can be easily positioned in the observation beam path at different positions, depending on what is required each time.

A second attenuating element can be fastened in a movable manner, for example, on the holder between the objective and the first attenuating element Light beams reflected from the cornea of the eye are blocked by the second attenuating element, so that these also can be filtered out of the observation beam bundle(s) and the observer of the eye being examined has an optimal view onto the eye. Due to the fact that the second attenuating element can be fastened so that it can move along the holder between the objective and the first attenuating element, the optical device according to the invention can easily be adjusted to any cornea of an eye being examined. That is, the light beams falling on a specific eye are reflected differently due to the different curvatures of the corneas of different eyes. The differently reflected light beams can be blocked in a particularly simple manner by the second attenuating element that is mounted in a movable manner. The additional adjustability of the transparency of the regions of the second attenuating element, particularly from a transparent state to a diffuse state and vice versa, creates a particularly simple and good blocking of the light beams reflected by the cornea.

The holder is preferably a rail, a slide guide, a rod or the like, along which the at least one attenuating element can be guided. The at least one attenuating element can be fastened at any desired position along the holder by means of fastening elements on the holder and corresponding fastening elements on the attenuating element. The height of the at least one attenuating element can be adjusted mechanically or electrically.

Of advantage is an optical device, in which the at least one attenuating element can be fastened on the holder so that its inclination can be changed. The attenuating element can thus be placed orthogonally or inclined at an angle in the observation beam path of the optical device.

Further, an optical device is particularly preferred, in which the at least one attenuating element is disposed in the observation beam path of the optical device outside the intermediate image plane of the retina of the eye being observed. “Outside the intermediate image plane of the retina” means that the at least one attenuating element is disposed in the observation beam path of the optical device at a certain distance relative to the intermediate image plane of the retina. Due to the arrangement outside the intermediate image plane of the retina, the observed image of the retina is either not influenced or is only slightly influenced. The at least one attenuating element will not be disposed in the intermediate image plane of the object being examined, i.e., the region of interest, but rather preferably directly where light reflections arise and/or in the image plane or in the vicinity of the image plane of the site where light reflections are formed.

In addition, an optical device is advantageous, in which the at least one attenuating element is disposed in the observation beam path of the optical device in or near the intermediate image plane of the cornea of the eye being observed and/or above the intermediate image plane of the cornea of the eye being observed. The closer the at least one attenuating element is found to the intermediate image plane of the cornea, the sharper is the blocking of the light reflections on the cornea, i.e., the disrupting light reflections can be more optimally blocked by the at least one attenuating element.

Due to the different curvatures of the cornea of different eyes, the intermediate image plane, i.e., the plane lying orthogonal to the optical axis of the optical device, in which the image of the cornea is found, is disposed at different distances to the optical element, particularly to a lens, and to the objective. Due to the fact that the at least one attenuating element is mounted in the holder in a movable manner, this element can be easily moved into or close to the respective intermediate image plane of the cornea. The light beams reflected on the cornea of the eye observed with the optical device are then blocked particularly simply by the at least one attenuating element. The intermediate image plane of the cornea is usually imaged clearly at a distance to the intermediate image plane of the retina of the eye, so that the at least one attenuating element is provided in the observation beam path at a sufficient distance from the intermediate image plane of the retina. In order to separate the image planes of the cornea and the retina, an optical element or an optical arrangement with a minimum depth of field is preferably employed.

It is preferred if an attenuating element is disposed directly in or in the vicinity of the intermediate image plane of the cornea that is produced by the opthalmoscopic magnifier. Then the regions of the attenuating element that bring about the suppression of reflections can be particularly small and can be controlled with a smaller degree of transparency (up to opacity). In this way, light reflections at the opthalmoscopic magnifier can be particularly effectively blocked.

An optical device, in which the lens can be fastened to the holder in a detachable manner, represents a very flexible optical device, since different lenses with different strengths can be fastened to the optical device in this way. In order to obtain very detailed views, for example, of the fundus of the eye, lenses with diopter numbers of more than 40 diopters can be employed. If only a coarse examination of an eye is to be made, lenses with diopter numbers of less than 40 diopters may be attached to the holder.

In addition, an optical device is advantageous, in which the optical element or the optical arrangement can be adjusted relative to its inclination. Due to the possible inclinations of the optical element, particularly a lens, on the holder, the light beams reflected on the two surfaces of the lens can be converged into one point. The convergence occurs preferably in the plane in which the at least one attenuating element is disposed. In this way, the disruptive light beams reflected on the lens are particularly easily blocked. The optical element or the optical arrangement can be inclined by mechanical or electrical adjustment.

In another preferred embodiment of the optical device, a lighting source is mounted in a movable manner on the holder. Different illuminations of the eye can be readily provided, due to the movable attachment of the lighting source to the holder. Therefore, the illumination beam bundle can be guided in cylindrical, conical or crossed form onto the lens of the optical device.

The at least one attenuating element preferably represents an electro-optical switch, which can be controlled electronically. A rapid switching of the respective regions of the at least one attenuating element between the different transparency states can be assured by the electronic control of the at least one attenuating element. In addition to the state of complete blocking of the beams, i.e., the diffuse state, or the complete passage of beams, i.e., the transparent state, any transparency state between the two extremes may also be provided. This is not possible with the use of a diaphragm, as is known from the prior art. Timed runs may also be adjusted in advance, so that a timed pattern for the states of the at least one attenuating element or individual regions of the at least one attenuating element can be provided.

The at least one attenuating element is preferably designed as a liquid crystal element, a polymer shutter diaphragm, an LCD matrix or an electrowetting matrix. An electronically switchable liquid crystal element, a polymer shutter diaphragm, an LCD matrix or an electrowetting matrix are particularly advantageous, since the latter can be reliably brought into two different transparency states, on the one hand, and they possess a very high response speed with the control, on the other hand. As a polymer shutter, an optical element that operates on the basis of electronically controllable light scatter is particularly designated. This optical element is controlled by an external electrical field, wherein the optical element is highly transparent if the electrical field is turned off, due to the corresponding alignment of the crystals, whereas the optical element is provided with a higher degree of opacity and thus with a high scatter capacity when the electrical field is applied. Polymer shutters operate with nonpolarized light and make possible a high transmission over the entire visible region. Polymer shutters, which have a reaction time in the sub-millisecond range, can be employed advantageously.

The present invention is not limited to a specific embodiment of a polymer shutter. One possible embodiment may be constituted, for example, by a pair of glass disks with an active layer disposed in between, whereby the active layer has free liquid crystal molecules. This can be accomplished by a photopolymerisation of liquid crystal polymer molecules in the presence of conventional liquid crystals.

In the case of the polymer shutter, for example, transparent electrodes can be used for introducing the electrical field.

The voltage with which the polymer shutter can be loaded may lie at 200 V, for example, whereby this represents the difference between the maxima of a voltage curve. For the operation of such a polymer shutter or for several such polymer shutters, only additional electrical connections must be provided on the polymer shutter(s).

“Control or activation of the at least one attenuating element” in the sense of the invention is to be understood as the placing of the at least one attenuating element or the individual regions of the at least one attenuating element into another transparency state, particularly into the diffuse state. In an attenuating element that operates electronically, control thus means the application of a necessary voltage for adjusting the transparency state.

Preferably, the optical device has an actuating device for controlling the at least one attenuating element. This actuating device may be a switch, for example, by means of which the at least one attenuating element or regions of the at least one attenuating element can be activated and deactivated. Preferably, a specific switch is assigned to each attenuating element, in order to be able to make possible a separate control of each attenuating element.

An optical device in which the lens has a strength of more than 40 diopters is preferred. This is of advantage for detailed observations of the eye.

Further, an optical device is preferred that has an analog or computer-supported regulating unit which evaluates the intensity of reflections in a locally resolved manner and suppresses the reflections automatically and at least partially within a control loop. A manual adjustment of the parameters of the attenuating element takes time. It happens that the patient's eye and/or the patient's head moves during the surgery. Adjustment must then be performed many times. This disrupts the work flow. The full ergonomics of freedom from reflections can thus perhaps be attained only with automatic adjustment.

The object is further achieved by an optical device for the examination of an eye, with at least one objective disposed facing the eye, at least one lighting source for illuminating the eye, an optical element and/or an optical arrangement disposed between the objective and the eye being examined, which optical element or arrangement produces an intermediate image of the plane examined within the eye, which is observed with the objective, whereby the optical device has an analog or computer-supported regulating unit which evaluates the intensity of reflections in a locally resolved manner and suppresses the reflections automatically and at least partially within a control loop. Time can be saved by an automatic adjustment of the parameters of the attenuating element. In addition, the examination of the eye is simplified by an automatic suppression of reflections, in particular when the patient's eye and/or the patient's head moves during the surgery. A manual adjustment must be performed many times. This disrupts the work flow. The full ergonomics of freedom from reflections can thus perhaps be attained only with automatic adjustment.

The object of the invention is achieved further by a method for blocking of light reflections in the observation beam path of an optical device, which is designed for the examination of an eye, particularly the retina of an eye, in which the blocking of light reflections in the observation beam path is produced by means of an above-described optical device according to the invention. Due to the fact that individual regions/blocks of the attenuating element can be moved into at least two different transparency states, light reflections from the optical element, particularly from a lens, as well as from the cornea of the eye being examined can be blocked in a targeted manner. The regions of the at least one attenuating element darken the places where the light reflections run in the beam bundle of the observation beam path, so that the light reflections can no longer reach the eye of the observer. That is, the regions/blocks of the attenuating element are switched as a function of the light intensity of the individual observation beams of the observation beam path. When a pre-set light intensity is exceeded, the transparency of the respective regions/blocks of the attenuating element is changed. When the light intensity is high, the regions/blocks of the attenuating element will switch to “diffuse”, so that the bright reflected light beams will be blocked. With low or normal light intensities, all regions/blocks of the attenuating element remain switched to “transparent”. This can be done manually, but also automatically, whereby the observed image is decoupled e.g, by capturing with a camera and is evaluated by software and then a control is initiated to suppress reflections. Such an arrangement takes into account the unavoidable eye and/or head movements of the patient.

A method for blocking light reflections in the observation beam path of an optical device is preferred, in which the optical device, particularly the objective of the optical device, is operated with a small depth of field. In this way, intermediate images or intermediate image planes in the observation beam path can be easily separated.

The use of an above-described optical device according to the invention for investigating an eye, particularly the retina of the eye, makes possible an optimal observation of the eye being examined for the observer or the operating surgeon. By the use of at least one locally-resolved attenuating element, disturbing light reflections, which come from the optical element, particularly the lens, of the optical device, as well as from the eye being examined, particularly the cornea of the eye, can be blocked in a targeted manner.

The optical device according to the invention represents an observation apparatus, particularly an opthalmoscope or a microscope, particularly having a slit lamp. The optical element is preferably a lens of high refractive power.

The invention will be explained below in more detail based on embodiment examples with reference to the attached drawings. Here:

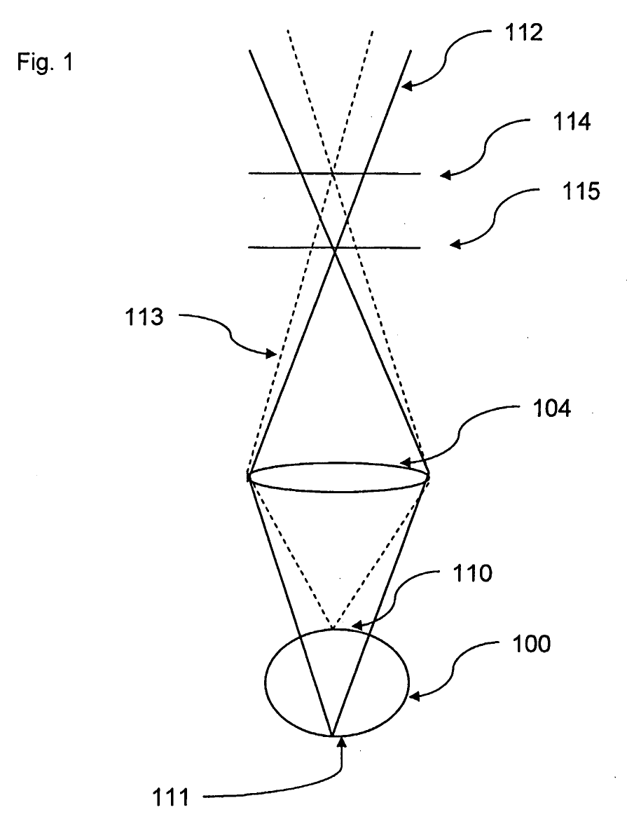

FIG. 1 shows a schematic representation of the beam paths reflected from the cornea and the retina of the eye being examined;

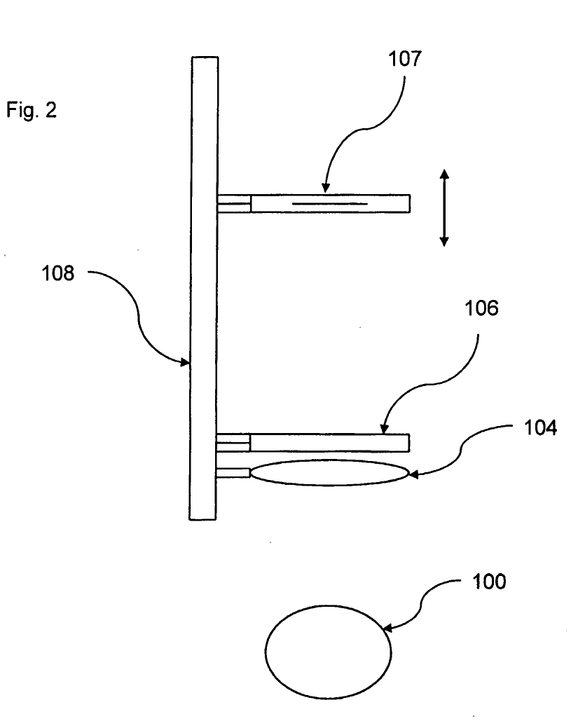

FIG. 2 shows schematically an embodiment of the holder with attenuating element and optical element of an optical device according to the invention;

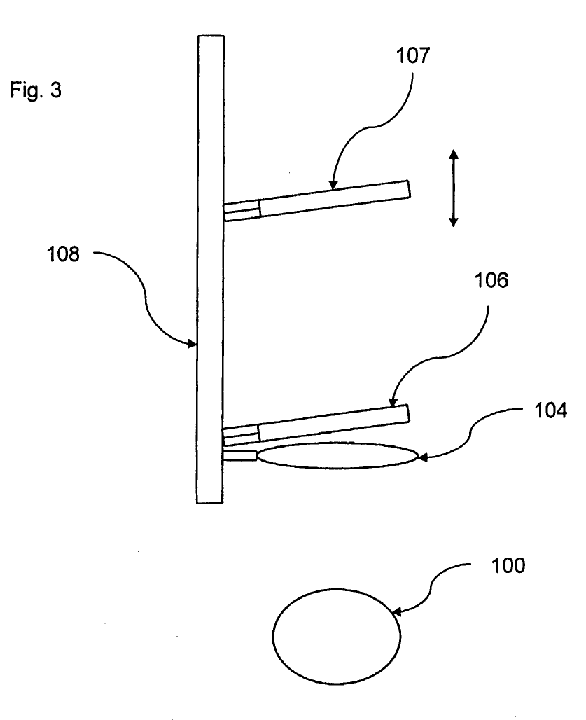

FIG. 3 shows schematically another embodiment of the holder, of the at least one attenuating element and of the optical element of the optical device according to the invention;

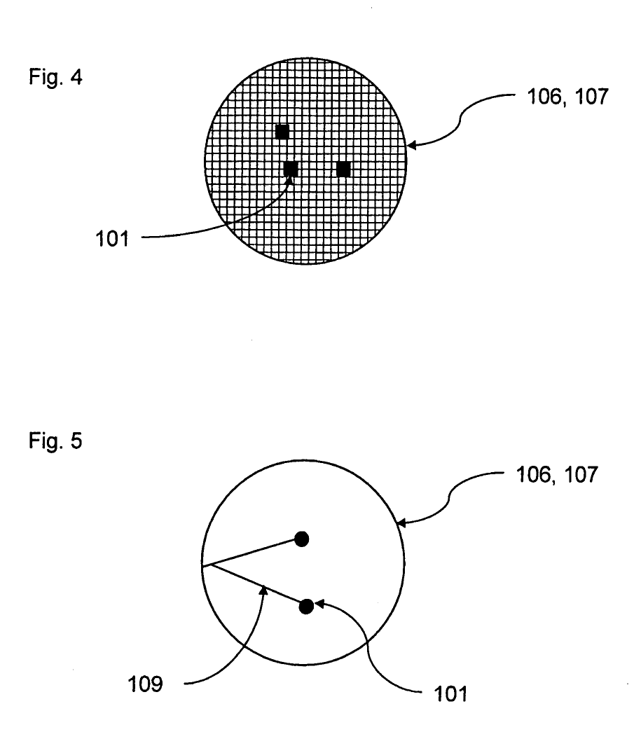

FIG. 4 shows an attenuating element with blocked regions;

FIG. 5 shows an attenuating element with pointers.

FIG. 1 shows a schematic representation of the beam paths 113 or 112 reflected from the cornea 110 and the retina 111 of the eye being examined 100. The intermediate image plane 114 of the cornea 110 lies between the optical element 104, here a lens, and the objective (not shown). The intermediate image 115 of the retina 111 lies between the intermediate image plane 114 of the cornea 110 and the lens 104. In order to separate the intermediate image plane 115 of the retina 111 from the intermediate image plane of the cornea 110, an optical device, in particular an operating microscope or an opthalmoscope, having a small depth of field is preferably operated. Due to the illumination of the eye 100 by lighting sources disposed on the optical device, light beams, which are disruptive for the observer who is examining eye 100, are reflected on cornea 110. These light reflections must be blocked so that an improved result of examination can be attained.

FIG. 2 schematically shows an embodiment of holder 108, two attenuating elements 106, 107 and an optical element 104, here a lens, of an optical device according to the invention. The holder 108 sits on the lower end of the optical device facing eye 100. Lens 104 is disposed on the end of holder 108 facing eye 100, so that the lens 104 preferably sits at a distance of approximately 50 mm to 150 mm in front of the eye when eye 100 is examined. The objective (not shown) of the optical device preferably sits at a distance of 400 mm to 600 mm from eye 100. A first attenuating element 106 is disposed on holder 108 on the side of lens 104 which is not facing eye 100. The first attenuating element 106 preferably sits directly in front of the lens 104 in order to directly block the disruptive light beams reflected on the surfaces of lens 104. The second attenuating element 107 sits at a greater distance from lens 104 than the first attenuating element 106. The second attenuating element 107 preferably blocks the disruptive light beams reflected from the cornea 110 of eye 100. In order to assure an optimal blocking of the reflected light beams, the second attenuating element 107 is fastened so that it can move along holder 108. In this way, the second attenuating element 107 can be placed precisely in the intermediate image plane 114 of cornea 110 of any eye 100. Since the cornea 110 is different for each eye 100, an optimal adaptation to each eye 100 is assured.

Individual regions or several regions of the first attenuating element 106 and of the second attenuating element 107 can thus be switched between at least two transparency states, particularly a highly transparent state and a scatter state. That is, in the scatter state, also called the diffuse state, the corresponding regions block the reflected light beams. The first attenuating element 106 and the second attenuating element 107 assume the function of partial shutter diaphragms or of light traps. In the transparent state, the attenuating elements 106, 107 or the regions of the attenuating elements 106, 107 do not hinder the observation beam path(s). The two attenuating elements 106, 107 represent so-called block matrixes. That is, the cross-sectional surfaces of the attenuating elements 106, 107 are divided into a plurality of blocks, whereby each individual block can be switched into at least two transparency states, particularly into a fully transparent state and into a diffuse state.

FIG. 3 shows another example of embodiment of a holder 108 with attenuating elements 106, 107 and a lens 104 of the optical device according to the invention. In contrast to FIG. 2, the two attenuating elements 106, 107 are disposed in an inclined manner. That is, the two attenuating elements 106, 107 are variable in their inclination. The attenuating elements 106, 107 can be inclined in such a way that they themselves do not contribute to a disruptive light reflection.

FIGS. 4 and 5 each show a cross section through an attenuating element 106, 107. FIG. 4 shows in black the diffuse regions 101 of the attenuating elements 106, 107. The attenuating elements 106, 107 are divided into a plurality of regions/blocks. That is, each attenuating element 106, 107 represents a so-called block matrix. Each individual block of each attenuating element 106, 107 can be switched into at least two different transparency states, in particular from a transparent state into a diffuse state. Each time depending on where the disruptive light reflections run in the observation beam path of the optical device, the regions/blocks can be switched so that precisely these disruptive light reflections will be blocked.

The attenuating element 106, 107 may also be designed in such a way that it has one or more pivotable pointers 109, wherein the end piece or the end pieces of the pointer(s) has (have) the size of one or more raster(s); see FIG. 5. Depending on what is needed in each case, the pointers 109 can cover targeted regions of an attenuating element 106, 107 and thus block parts of an observation beam path.

### LIST OF REFERENCE NUMBERS

100 eye being examined
101 diffuse regions of an attenuating element
104 optical element (lens)
106 first attenuating element
107 second attenuating element
108 holder
109 pointer of an attenuating element
110 cornea
111 retina
112 beam path of the retina
113 beam path of the cornea
114 intermediate image plane of the cornea
115 intermediate image plane of the retina

1. An optical device for the examination of an eye, with at least one objective disposed facing the eye, at least one lighting source for illuminating the eye, an optical element and/or an optical arrangement disposed between the objective and the eye being examined, which optical element or arrangement produces an intermediate image of the plane examined within the eye, which is observed with the objective, is hereby characterized in that in the observation beam path of the optical device, at least one locally resolved attenuating element is disposed, which can be switched in a locally resolved manner into at least two transparency states.

2. The optical device according to claim 1, further characterized in that the at least one attenuating element is fastened so that it can be moved along a holder of the optical device.

3. The optical device according to claim 1, further characterized in that the at least one attenuating element is fastened on holder so that its inclination can be changed.

4. The optical device according to claim 1, further characterized in that the at least one attenuating element is disposed in the observation beam path of the optical device outside the intermediate image plane of the retina of the eye being observed.

5. The optical device according to claim 1, further characterized in that the at least one attenuating element is disposed in the observation beam path of the optical device in or near the intermediate image plane of the cornea of the eye being observed and/or above the intermediate image plane of the cornea of the eye being observed.

6. The optical device according to claim 1, further characterized in that the at least one attenuating element is disposed near/on the optical element on the side not facing the object or the eye.

7. The optical device according to claim 1, further characterized in that the at least one attenuating element is designed in a spectrally resolved manner.

8. The optical device according to claim 1, further characterized in that the inclination of the optical element and/or the optical arrangement can be adjusted.

9. The optical device according to claim 1, further characterized in that the lighting source can be mounted in a movable manner on holder.

10. The optical device according to claim 1, further characterized in that the at least one attenuating element is an electro-optical switch.

11. The optical device according to claim 1, further characterized in that the at least one attenuating element is designed as a liquid crystal element, a polymer shutter diaphragm, an LCD matrix or an electrowetting matrix.

12. The optical device according to claim 1, further characterized in that at least two attenuating elements can be controlled separately.

13. The optical device according to claim 1, further characterized in that the optical device has an actuating device for controlling the at least one attenuating element.

14. The optical device according to claim 1, further characterized in that the optical element and/or the optical arrangement has a strength of more than 40 diopters.

15. The optical device according to claim 1, further characterized in that it has an analog or computer-supported regulating unit which evaluates the intensity of reflections in a locally resolved manner and suppresses the reflections automatically and at least partially within a control loop.

16. An optical device for the examination of an eye, with at least one objective disposed facing the eye, at least one lighting source for illuminating the eye, an optical element and/or an optical arrangement disposed between the objective and the eye being examined, which optical element or arrangement produces an intermediate image of the plane examined within the eye, which is observed with the objective, is hereby characterized in that it has an analog or computer-supported regulating unit which evaluates the intensity of reflections in a locally resolved manner and suppresses the reflections automatically and at least partially within a control loop.

17. A method for blocking light reflections in the observation beam path of an optical device, which is designed for the examination of an eye, in particular the retina of an eye, is hereby characterized in that the light reflections in the observation beam path are blocked with an optical device according to claim 1 or claim 16.

18. The method for blocking light reflections in the observation beam path of an optical device according to claim 17, further characterized in that the optical device, in particular the objective of the optical device, is operated with a small depth of field.

19. Use of an optical device according to claim 1 or claim 16 for the examination of an eye, particularly of the retina of the eye.
